(19) United States
(12) Patent Application Publication
(10) Pub. No.: US 2006/0115446 A1
(43) Pub. Date: Jun. 1, 2006

(54) Thickened hair composition comprising at least one high-molecular-weight polyurethane

(76) Inventors: Isabelle Rollat-Corvol, Paris (FR); Pascale Cothias, Montigny le Bretonneau (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/260,432

(22) Filed: Oct. 28, 2005

Related U.S. Application Data
(60) Provisional application No. 60/645,750, filed on Jan. 24, 2005.

(30) Foreign Application Priority Data
Oct. 28, 2004 (FR) 04 52468

(51) Int. Cl. A61K 8/87

(52) U.S. Cl. 424/70.17

(57) ABSTRACT

The present disclosure relates to a hair cosmetic composition, such as a gel, comprising at least one high-molecular-weight polyurethane polymer and at least one thickener. The present disclosure also relates to a hair cosmetic method comprising the application of said composition to the hair and its use to fix and/or hold the hairstyle.

This application claims benefit of U.S. Provisional Application No. 60/645,750, filed Jan. 24, 2005, the contents of which are incorporated herein by reference. This application also claims benefit of priority under 35 U.S.C. § 119 to French Patent Application No. 04 52468 filed Oct. 28, 2004, the contents of which are also incorporated herein by reference.

The present disclosure relates to a hair cosmetic composition, for instance a gel, comprising at least one high-molecular-weight polyurethane polymer and at least one thickener. The present disclosure also relates to a cosmetic hair method to fix and/or hold the hairstyle comprising the application of the composition as disclosed herein to the hair.

As used herein, the expression “high-molecular-weight polyurethane” is understood to mean a polyurethane with a number-average molecular weight ranging from 400,000 to 5,000,000 g/mol.

As used herein, the term “gel” is understood to mean a composition which is originally liquid, which has been thickened using an agent having thickening and/or gelling properties, having a minimal viscosity of greater than or equal to 160 cps, such as 250 cps at 25° C. (Rheomat 180 viscometer—No. 2 rotor—reading after 30 s).

As used herein, the term “thickener” is understood to mean any compound capable of increasing the viscosity of a cosmetic composition.

Products for fixing and/or holding the hairstyle are known and are provided in various forms, for instance in the form of gels. While these often allow lasting fixing of the hairstyle, they nevertheless can have various disadvantages.

For example, the polymers commonly used as fixing agents in gels do not always make it possible to preserve the shaping/styling of the hair when the latter is brought into contact with liquid water for a prolonged period, such as for example on contact with rain, perspiration or during swimming in the sea or swimming pools.

The known gels can also be criticized for leading to a hairstyle which is poorly resistant to the moisture in the air.

To remedy the drawbacks discussed above, it has already been proposed to produce hair cosmetic products comprising a fixing polymer and a thickening agent with optional additives. Nevertheless, the compositions known up until now comprising these constituents still are not always completely satisfactory.

Thus, the present disclosure relates to a hair cosmetic composition, in gel form, which has improved properties, for example, when exposed to liquid water when compared with the prior art compositions.

Surprisingly and unexpectedly, it has been discovered that by judiciously selecting the at least one fixing polymer, it is possible to solve at least one of the problems discussed above.

Accordingly, one aspect of the present disclosure is a cosmetic composition provided in the form of a gel and comprising, in a cosmetically acceptable medium, at least one high-molecular-weight polyurethane with a number-average-molecular-weight ranging from 400,000 to 5,000,000 g/mol and at least one thickener.

These compositions can make it possible to fix and shape the hair into hairstyles and also to preserve the shaping of the hairstyles when the latter are brought into contact with liquid water for a prolonged period of time. They can also make it possible to obtain a hairstyle which is resistant to the moisture in the air.

Another aspect of the present disclosure relates to a cosmetic method for hair, such as a method for fixing and/or holding the hairstyle using the composition as disclosed herein.

Yet another aspect of the present disclosure relates to the use of this composition in a hair cosmetic formulation intended, for instance, for holding and/or for shaping the hairstyle.

As used herein, the expression “prolonged period,” is understood to mean bringing into contact with liquid water for a minimum period of one minute, for example 10 minutes, such as 20 minutes.

As used herein, the at least one “high-molecular-weight polyurethane” and the at least one “thickener” are compounds which are distinct from each other.

High-Molecular-Weight Polyurethanes

As used herein, the term “polyurethane” is understood to mean polycondensates comprising at least one polyurethane block. They are described, for instance, in European Patent Nos. EP 0 751 162, EP 0 637 600, EP 0 648 485, EP 0 656 021, and EP 0 619 111, French Patent No. FR 2 743 297, and PCT Application Publication No. WO 94/03510.

The polyurethanes used in accordance with the present disclosure may be soluble in the cosmetically acceptable aqueous medium, for example, after neutralizing with an organic or inorganic base, or may form a dispersion in this medium. The dispersion may then comprise at least 0.05% of surfactant allowing the dispersion and maintaining the nonassociative polyurethane in dispersion.

According to the present disclosure, it is possible to use any type of surfactant in the dispersion, for instance, in one embodiment, a nonionic surfactant is used. The average size of the particles of the at least one nonassociative polyurethane in the dispersion for example, can range from 0.1 μm to 1 μm.

By way of non-limiting example, the at least one high-molecular-weight polyurethane may be formed by an arrangement of blocks, this arrangement being obtained, for instance, from:
(1) at least one compound which comprises at least two active hydrogen atoms per molecule;
(2) at least one diol, or a mixture of diols comprising acid functional groups or salts thereof; and
(3) at least one di- or polyisocyanate.

By way non-limiting example, the compounds (1) can be chosen from the group comprising diol, diamine, polyesterol, and polyetherol groups and mixtures thereof.

Also among the compounds (1) which may be used, further non-limiting mention may be made of linear polyethylene glycols and polypropylene glycols, for instance, those which are obtained by the reaction of ethylene or propylene oxide with water or of diethylene or dipropylene glycol in the presence of sodium hydroxide as catalyst. These polyalkylene glycols generally have a molecular mass ranging from 600 to 20,000.

Other organic compounds that may be used include those which have mercapto, amino, carboxyl or hydroxyl groups. Among these compounds, non-limiting mention may be made of polyhydroxylated compounds such as polyether diols, polyester diols, polyacetal diols, polyamide diols, polyester-polyamide diols, poly(alkylene ether) diols, polythioether diols and polycarbonate diols.

Among the polyether diols that may be used, for example, non-limiting mention may be made of the products of condensation of ethylene oxide, propylene oxide or tetrahydrofuran, their graft or block products of copolymerization or condensation such as the mixtures of condensates of ethylene and propylene oxide, and the products of polymerization of olefins, at high pressure, with the condensates of alkylene oxide. Further appropriate polyether diols include, by way of non-limiting example, those prepared by condensation of alkylene oxides and polyhydric alcohols, such as ethylene glycol, 1,2-propylene glycol and 1,4-butanediol.

The polyester diols, polyester amides, polyamide diols can be, for instance, saturated and can be obtained for example from the reaction of saturated or unsaturated polycarboxylic acids with polyhydric alcohols, diamines or polyamines. To prepare these compounds, it is possible to use, for example, adipic acid, succinic acid, phthalic acid, terephthalic acid and maleic acid. Appropriate polyhydric alcohols for preparing the polyesters include, for example, polyethylene glycol, 1,2-propylene glycol, 1,4-butanediol, neopentyl glycol and hexanediol. It is also possible to use amino alcohols, for example ethanolamine. Appropriate diamines for preparing amide polyesters include ethylenediamine and hexamethylenediamine.

Appropriate polyacetals may be prepared, for example, from 1,4-butanediol and hexanediol and formaldehyde. Appropriate polythioethers may be prepared, for example, by condensation reaction between thioglycols alone or in combination with other glycols such as ethylene glycol, 1,2-propylene glycol or with other polyhydroxylated compounds. The polyhydroxylated compounds already comprising urethane groups, natural polyols, which may be further modified, for example, castor oil and carbohydrates may also be used.

In one embodiment, the group (1) compound can be a polyesterol, for instance, a polyester diol formed by the reaction of at least one (di)polyol (1ₐ) and at least one acid (1_{b}). The (di)polyol (1ₐ) can be chosen for example from the group comprising neopentyl glycol, 1,4-butanediol, hexanediol, ethylene glycol, diethylene glycol, propylene glycol, butylene glycol, neopentyl glycol and (di)polyethylene glycol. The acid (1_{b}) can be chosen for example from the group comprising phthalic acid, isophthalic acid, adipic acid and (poly)lactic acid.

Among the compounds (2), that may be used, non-limiting mention may be made of hydroxycarboxylic acid such as dimethylolpropanoic acid (DMPA) or a 2,2-hydroxymethylcarboxylic acid. In general, the compound (2) is useful as a coupling block. In one embodiment of the present disclosure, at least one compound (2) is chosen from those comprising at least one poly(α,α-dihydroxylated carboxylic acid).

Further non-limiting mention among the compounds (2) may be made of those chosen from the group comprising 2,2-di(hydroxymethyl)acetic acid, 2,2-dihydroxymethylpropionic acid, 2,2-dihydroxymethylbutyric acid, and 2,2-dihydroxymethylpentanoic acid.

Among the di- or polyisocyanate compounds (3), non-limiting mention may be made of those chosen from the group comprising hexamethylene diisocyanate, isophorone diisocyanate (IDPI), tolylene diisocyanate, diphenylmethane 4,4′-diisocyanate (DPMD) and dicyclohexylmethane 4,4′-diisocyanate (DCMD), methylene di-p-phenyl diisocyanate, methylene bis(4-cyclohexyl isocyanate), toluene diisocyanates, 1,5-naphthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4- and 2,6-toluene diisocyanates, 2,2′-dichloro-4,4′-diisocyanatodiphenylmethane, 2,4-dibromo-1,5-diisocyanatonaphthalene, butane 1,4-diisocyanate, hexane 1,6-diisocyanate and cyclohexane 1,4-diisocyanate.

The at least one nonassociative high-molecular-weight polyurethane may be formed with the aid of at least one additional compound (4) which serves in general to extend its chain. These compounds (4) may be chosen from the group comprising, by way of non-limiting example, saturated or unsaturated glycols such as ethylene glycol, diethylene glycol, neopentyl glycol, triethylene glycol; amino alcohols such as ethanolamine, propanolamine, butanolamine; heterocyclic, aromatic, cycloaliphatic and aliphatic primary amines; diamines; carboxylic acids such as aliphatic, aromatic and heterocyclic carboxylic acids such as oxalic, succinic, glutaric, adipic, sebacic and terephthalic acids; and aminocarboxylic acids. In one embodiment of the present disclosure, the compounds (4) are aliphatic diols.

The high-molecular-weight polyurethanes used according to the present disclosure may also be further formed from at least one additional compound (5) having a silicone backbone such as polysiloxanes, polyalkylsiloxanes or polyarylsiloxanes, for instance polyethylsiloxanes, polymethylsiloxanes and polyphenylsiloxanes, optionally comprising hydrocarbon chains grafted onto the silicon atoms.

In another embodiment of the present disclosure, the high-molecular-weight polyurethanes used comprise a basic repeating unit of formula (I):
—O—B—O—CO—NH—R—NH—CO— (I)
wherein:

B is chosen from divalent C₁ to C₃₀ hydrocarbon groups, being unsubstituted or substituted with a group comprising at least one carboxylic acid functional group and/or at least one sulphonic acid functional group, the said carboxylic and/or sulphonic acid functional groups being in a free form or partially or completely neutralized with an inorganic or organic base, and

R is a divalent group chosen from C₁ to C₂₀ aliphatic, C₃ to C₂₀ cycloaliphatic and C₆ to C₂₀ aromatic hydrocarbon groups such as, for example, the C₁ to C₂₀ alkylene, C₆ to C₂₀ arylene and C₃ to C₂₀ cycloalkylene groups, or combinations thereof, these groups being substituted or unsubstituted.

In yet another embodiment of the present disclosure, the group R can be chosen from the groups of formulae: 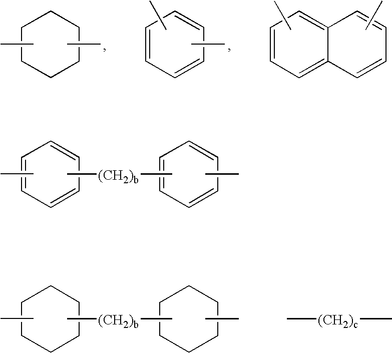
in which b is an integer ranging from 0 to 3, and c is an integer ranging from 1 to 20, such as from 2 to 12.

For example, the group R can be chosen from hexamethylene, 4,4′-biphenylenemethane, 2,4- and/or 2,6-tolylene, 1,5-naphthalene, p-phenylene and methylene-4,4-biscyclohexyl groups and the divalent group derived from isophorone.

The high-molecular-weight polyurethane used in accordance with the present disclosure may additionally comprise at least one polysiloxane block.

The high-molecular-weight polyurethanes of the present disclosure can be, for example, nonassociative, which as used herein means that the polyurethane has a sturcture not comprising an alkyl or alkenyl chain having more than 10 carbon atoms.

The at least one high-molecular-weight polyurethane can be present in the composition according to the present disclosure in an amount ranging from 0.05% to 40% by weight, such as from 0.1% to 20% by weight, and for instance ranging from 1% to 8% by weight, relative to the total weight of the composition for the cosmetic treatment of keratin materials.

In one embodiment of the present disclosure, the at least one high-molecular-weight polyurethane has a molecular mass ranging from 400,000 g/mol to 3,000,000 g/mol, for instance from 1,000,000 g/mol to 2,500,000 g/mol.

In at least one embodiment of the present disclosure, the at least one high-molecular-weight polyurethane is AVALURE UR-450 marketed by the company Noveon, which is an anionic copolymer formed of PPG-17 (polypropylene glycol with a number of units n=17)/IPDI (isophorone diisocyanate)/DMPA (dimethylolpropionic acid). In an aqueous medium, it exists in the form of a dispersion. Its molecular weight is 1,830,000 g/mol.

Thickeners

The composition also comprises, for example, as a percentage relative to the weight of the composition, at least one thickener in an amount ranging from 0.01% to 20%, such as from 0.05% to 10%.

By way of non-limiting example, the thickening agent can be chosen from natural or natural modified polymers (i), crosslinked copolymers of acrylic and/or methacrylic acid (ii), crosslinked thickening polyacrylamides (iii) and associative polymers comprising at least one hydrophilic unit and at least one fatty chain (iv).

As used herein, the expression “natural modified thickener (i)” is understood to mean any thickening polymer obtained by simple chemical modification from the natural polymer itself.

There may be mentioned as natural or natural modified thickeners (i) which are suitable for the present disclosure, by way of non-limiting example, cellulose gum, scleroglucan gum, gellan gum, rhamsan gum, alginates, karaya gum, carob flour, modified or unmodified guar gums and derivatives thereof, such as hydroxypropylguar. Guar gum and its derivatives (hydroxypropylguar), and gums of natural microbial origin (xanthan gum and scleroglucan gum).

Among the crosslinked copolymers of acrylic and/or methacrylic acid (ii) that can be used, non-limiting mention may be made of, for instance, acrylic acid/ethyl acrylate copolymers and carboxyvinyl polymers. Such polymers include, by way of non-limiting example, the “carbomers” (CTFA) sold by the company Goodrich under the name CARBOPOL.

Among the crosslinked thickening polyacrylamides (iii) that may be used, non-limiting mention may be made of those chosen from:

crosslinked homopolymers of 2-acrylamido-2-methylpropanesulphonic acid,

optionally crosslinked copolymers of acrylamide and ammonium acrylate,

crosslinked copolymers of acrylamide (or methacrylamide) and methacryloyloxyethyltrimethylammonium chloride,

partially or completely neutralized, crosslinked copolymers of acrylamide and 2-acrylamido-2-methylpropanesulphonic acid.

Among the crosslinked acrylamide/ammonium acrylate copolymers that can be used in accordance with the present disclosure, non-limiting mention may be made of the acrylamide/ammonium acrylate ( 5/95 by weight) copolymers crosslinked with an olefinically polyunsaturated crosslinking agent such as divinylbenzene, tetraallyloxyethane, methylenebisacrylamide, diallyl ether, polyallyl polyglyceryl ethers or allyl ethers of alcohols of the sugar series, such as erythritol, pentaerythritol, arabitol, mannitol, sorbitol or glucose.

Similar copolymers are described and prepared, for example, in French Patent No. FR 2 416 723, and U.S. Pat. Nos. 2,798,053 and 2,923,692.

This type of crosslinked copolymer can be used for example in the form of a water-in-oil emulsion comprising about 30% by weight of the said copolymer, 25% by weight of paraffin oil, 4% by weight of a mixture of sorbitan stearate and a hydrophilic ethoxylated derivative and 41% by weight of water. Such an emulsion is marketed under the name “BOZEPOL C” by the company Hoechst.

Among the copolymers of acrylamide and of 2-acrylamido-2-methylpropanesulphonic acid used in accordance with the present disclosure, non-limiting mention may be made of copolymers crosslinked with an olefinically polyunsaturated compound, such as those mentioned above, and partially or completely neutralized with a neutralizing agent such as sodium hydroxide, potassium hydroxide, aqueous ammonia or an amine such as triethanolamine or monoethanolamine.

They may be prepared by copolymerizing acrylamide and sodium 2-acrylamido-2-methylpropanesulphonate by the free-radical route by means of initiators of the azobisisobutyronitrile type and by precipitation in an alcohol such as tert-butanol.

In one embodiment of the present disclosure, the copolymers obtained by copolymerization of 70% to 55 mol % of acrylamide and 30% to 45 mol % of sodium 2-acrylamido-2-methylpropanesulphonate are used. The crosslinking agent can be used at concentrations of 10⁻⁴ to 4×10⁻⁴ mol per mole of the mixture of monomers.

In one embodiment of the present disclosure, these particular copolymers are incorporated into the compositions of the present disclosure, for example, in the form of water-in-oil emulsions comprising from 35% to 40% by weight of this copolymer, from 15% to 25% by weight of a mixture of C₁₂-C₁₃ isoparaffin hydrocarbons, from 3% to 8% by weight of polyethylene glycol lauryl ether comprising 7 moles of ethylene oxide and water. Such an emulsion is marketed under the name “SEPIGEL 305” by the company Seppic.

In another embodiment, the crosslinked copolymer of acrylamide and methacryloyloxyethyltrimethylammonium chloride used according to the present disclosure can be, for example, a copolymer obtained by copolymerization of acrylamide and dimethylaminoethyl methacrylate quaternized with methyl chloride, followed by crosslinking with an olefinically unsaturated compound, such as methylenebisacrylamide.

The non-crosslinked copolymers of methacrylamide and methacryloyloxyethyltrimethylammonium chloride can be, for example, the products sold under the trade names ROHAGIT KF 400 and KF 720 by the company Rhom and Haas.

Among the associative polymers (iv) of the present disclosure, non-limiting mention may be made of nonionic, cationic, anionic or amphoteric polymers. They may belong, for example, to the polyurethane class.

The associative polyurethanes are block copolymers comprising in the chain both hydrophilic blocks most often of a polyoxyethylenated nature and hydrophobic blocks which may be aliphatic linkages alone and/or cycloaliphatic and/or aromatic linkages.

For example, in one embodiment, these polymers can comprise at least two lipophilic hydrocarbon chains having from C₆ to C₃₀ carbon atoms, separated by a hydrophilic block, the hydrocarbon chains may be pendant chains or chains at the end of a hydrophilic block. For instance, it is possible for at least one pendant chain to be provided. In addition, the polymer may comprise at least one hydrocarbon chain at one end or at both ends of a hydrophilic block.

The polymers may be block polymers in triblock or multiblock form. The hydrophobic blocks may therefore be at each end of the chain (for example: triblock copolymer comprising a hydrophilic central block) or distributed both at the ends and inside the chain (multiblock copolymer for example). The polymers may also be graft or star-shaped polymers.

In one embodiment of the present disclosure, the polymers are triblock copolymers in which the hydrophilic block is a polyoxyethylenated chain comprising from 50 to 1000 oxyethylenated groups. In general, the associative polyurethanes comprise a urethane bond between the hydrophilic blocks, hence the name.

By extension, polymers in which the hydrophilic blocks are linked by other chemical bonds to the lipophilic blocks are also among the associative polyurethanes.

By way of non-limiting example of the associative polymers which can be used in the present disclosure, there may be mentioned the polymer C₁₆-EO₁₂₀-C₁₆ sold by the company Huls (under the name SERAD FX1100, a molecule containing a urethane functional group and having a weight-average molecular weight of 1300), EO being an oxyethylenated unit. Among the associative polymers, non-limiting mention may also be made of RHEOLATE 205 comprising a urea functional group sold by the company Rheox or RHEOLATE 208 or 204. These associative polyurethanes are sold in pure form.

The product DW 1206B from Rohm & Haas comprising a C₂₀ alkyl chain and comprising a urethane bond, sold at 20% as dry matter content in water, may also be mentioned in a non-limiting manner.

It is also possible to use solutions or dispersions of these polymers, for instance, in water or in an aqueous-alcoholic medium. By way of non-limitng example of such polymers, there may be mentioned SERAD FX1010 and SERAD 1035 sold by the company Huls, RHEOLATE 255, RHEOLATE 278 and RHEOLATE 244 sold by the company Rheox. It is also possible to use the product DW 1206F and DW 1206J, and ACRYSOL RM 184 or ACRYSOL 44 from the company Rohm & Haas.

In one embodiment of the present disclosure, the polymers which can be used are those described in the article by G. Fonnum, J. Bakke and Fk. Hansen—Colloid Polym. Sci. 271, 380,389 (1993).

Among the associative polyurethanes (iv) that may be used as disclosed herein, non-limiting mention may be made of those chosen from:

the polyether-polyurethanes (a) capable of being obtained by polycondensation of at least three compounds comprising (i) at least one polyethylene glycol comprising from 50 to 500 moles of ethylene oxide, (ii) at least one C₈-C₃₀ fatty alcohol and (iii) at least one diisocyanate, and

the polyether-polyurethanes (b) capable of being obtained by polycondensation of at least three compounds comprising (i) at least one polyethylene glycol comprising from 50 to 500 moles of ethylene oxide, (ii) at least one C₈-C₃₀ fatty alcohol different from that of the polyether-polyurethane (a) and (iii) at least one diisocyanate.

In one embodiment of the present disclosure, polyether-polyurethanes (a) and (b) for which the polyethylene glycol comprises 150 or 180 moles of ethylene oxide can be used.

In another embodiment of the present disclosure, the polyether-polyurethanes (a) and (b) for which the diisocyanate is methylene bis(4-cyclohexyl isocyanate) can be used.

In still another embodiment, the composition according to the present disclosure is a composition as defined above, for which the polyether-polyurethane (a) is obtained by polycondensation of at least three compounds comprising a polyethylene glycol comprising 150 or 180 moles of ethylene oxide, stearyl (C18) alcohol and methylene bis(4-cyclohexyl isocyanate), and the polyether-polyurethane (b) is obtained by polycondensation of at least three compounds comprising a polyethylene glycol comprising 150 or 180 moles of ethylene oxide, decyl (C10) alcohol and methylene bis(4-cyclohexyl isocyanate).

Among the polyether-polyurethanes (a), there may be mentioned, in a non-limiting manner, the product sold by the company Rohm & Haas under the trade name:

ACULYN 46, which is a polycondensate comprising at least, as components, a polyethylene glycol comprising 150 or 180 moles of ethylene oxide, stearyl alcohol and methylene bis(4-cyclohexyl isocyanate) (SMDI), at 15% by weight in a matrix of maltodextrin (4%) and water (81%).

Among the polyether-polyurethanes (b), there may be mentioned, in a non-limiting manner, the product sold by the company Rohm & Haas under the trade name:

ACULYN 44, which is a polycondensate comprising at least, as components, one polyethylene glycol comprising 150 or 180 moles of ethylene oxide, decyl alcohol and methylene bis(4-cyclohexyl isocyanate) (SMDI), at 35% by weight in a mixture of propylene glycol (39%) and water (26%).

It is also possible to use, as associative polyurethane, Viscophobe DB 1000 (Union Carbide).

As other associative polymers which can be used, there may be mentioned, in a non-limiting manner, the (meth)acrylic acid/C10-C30 alkyl acrylate copolymers such as Pemulen TR1 from Goodrich.

The cosmetically acceptable medium can consist of water, or can comprise at least one cosmetically acceptable solvent such as alcohols or water-solvent mixtures, these solvents being, for instance, C₁-C₄ alcohols, aliphatic or aromatic ketones, esters of C₁ to C₈ short chain or C₉ to C₂₀ long chain acids and of C₁ to C₈ short chain or C₉ to C₂₀ long chain alcohols, pentane, heptane, polyols, polyol ethers and isodecane.

Among the alcohols, non-limiting mention may be made of ethanol and isopropanol. In one embodiment, ethanol is used.

The ratio between the concentration by weight of the at least one high-molecular-weight polyurethane and the concentration by weight of the at least one thickener, in the compositions in accordance with the present disclosure, can be present in an amount, for example, ranges from 0.01% to 100%, for instance from 0.05% to 20%, such as from 0.1% to 10%.

The composition of the present disclosure may also comprise at least one adjuvant chosen from anionic, cationic, nonionic and amphoteric surfactants; perfumes; screening agents; preservatives; proteins; vitamins; provitamins; polymers other than those of the present disclosure; vegetable, mineral and synthetic oils; polyols such as glycols or glycerine; silicones; fatty alcohols; and any other adjuvant conventionally used in cosmetic compositions.

Of course, persons skilled in the art will be careful to choose the possible adjuvant(s) to be added to the composition according to the present disclosure such that the beneficial properties intrinsically attached to the composition in accordance with the present disclosure are not, or not substantially, impaired by the addition envisaged.

The compositions in accordance with the present disclosure may be applied to the skin, the nails, the lips, the hair, the eyebrows and the eyelashes.

The compositions in accordance with the present disclosure are suitable for dry or wet hair, when used as hair styling products.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. The example that follows serve to illustrate the present disclosure without, however, being limiting in nature.

All the percentages are relative percentages by weight relative to the total weight of the composition and a.i. means active ingredient.

### EXAMPLE

A hair-styling gel according to the invention is prepared:

| | | |
|---|---|---|
| (a) Avalure UR 450 (Noveon) | 7% | a.i. |
| (b) Jaguar HP105 (Rhodia) | 1% | a.i. |
| (c) Q2-5220 (Dow Corning) | 1% | a.i. |
| (d) Water | qs 100% | a.i. |

The hair-styling gel according to the present disclosure had a long stability over time. The hair styling carried out with the gel according to the present disclosure remained intact after having been dipped for 30 minutes in a tank of soft water.

What is claimed is:

1. A cosmetic composition for the cosmetic treatment of keratin materials comprising, in a cosmetically acceptable medium,
at least one high-molecular-weight polyurethane with a number-average molecular weight ranging from 400,000 to 5,000,000 g/mol, and
at least one thickener,
wherein the cosmetic composition is provided in the form of a gel.

2. The cosmetic composition according to claim 1, wherein the at least one high-molecular weight polyurethane has a number-average molecular mass ranging from 400,000 to 3,000,000.

3. The cosmetic composition according to claim 2, wherein the at least one high-molecular-weight polyurethane has a number-average molecular mass ranging from 1,000,000 to 2,500,000.

4. The cosmetic composition according to claim 1, wherein the at least one high-molecular-weight polyurethane is nonassociative.

5. The cosmetic composition according to claim 1, wherein the at least one high-molecular-weight polyurethane comprises a basic repeating unit of formula (1):
—O—B—O—CO—NH—R—NH—CO— (I)
wherein:
B is chosen from divalent C₁ to C₃₀ hydrocarbon groups, unsubstituted or substituted with a group comprising at least one carboxylic acid functional group and/or at least one sulphonic acid functional group, the said carboxylic and/or sulphonic acid functional groups being in a free form or partially or completely neutralized with an inorganic or organic base, and
R is a divalent group chosen from substituted or unsubstituted C₁ to C₂₀ aliphatic, C₃ to C₂₀ cycloaliphatic and C₆ to C₂₀ aromatic hydrocarbon groups, or combinations thereof.

6. The cosmetic composition according to claim 4, wherein the at least one high-molecular-weight polyurethane is present in an amount ranging from 0.05% to 40% by weight, relative to the total weight of the composition.

7. The cosmetic composition according to claim 6, wherein the at least one high-molecular-weight polyurethane is present in an amount ranging from 0.10% to 20% by weight, relative to the total weight of the composition.

8. The cosmetic composition according to claim 1, wherein the at least one thickening agent is chosen from
natural or natural modified polymers (i),
crosslinked copolymers of acrylic and/or methacrylic acid (ii),
crosslinked thickening polyacrylamides (iii), and
associative polymers comprising at least one hydrophilic unit and at least one fatty chain (iv).

9. The cosmetic composition according to claim 8, wherein the natural or natural modified polymers (i) are chosen from xanthan gum, cellulose gum, scleroglucan gum, gellan gum, rhamsan gum, alginates, karaya gum, carob flour, guar gums and derivatives thereof.

10. The cosmetic composition according to claim 8, wherein the crosslinked copolymers of acrylic and/or methacrylic acid (ii) are chosen from acrylic acid/ethyl acrylate copolymers and carboxyvinyl polymers.

11. The cosmetic composition according to claim 8, wherein the crosslinked thickening polyacrylamides (iii) are chosen from:
crosslinked homopolymers of 2-acrylamido-2-methylpropanesulphonic acid,
optionally crosslinked copolymers of acrylamide and ammonium acrylate,
crosslinked copolymers of acrylamide (or methacrylamide) and methacryloyloxyethyltrimethylammonium chloride,
partially or completely neutralized, crosslinked copolymers of acrylamide and 2-acrylamido-2-methylpropanesulphonic acid.

12. The cosmetic composition according to claim 8, wherein the associative polymers (iv) are chosen from associative polyurethanes.

13. The cosmetic composition according to claim 1, wherein the at least one thickener is present in the composition in an amount ranging from 0.01% to 20% by weight, relative to the total weight of the composition.

14. The cosmetic composition according to claim 13, wherein the at least one thickener is present in the composition in an amount ranging from 0.05% to 10% by weight, relative to the total weight of the composition.

15. The composition according to claim 1, wherein the at least one high-molecular-weight polyurethane is present in the composition such that the ratio of its concentration by weight to the concentration by weight of the at least one thickener ranges from 0.01% to 100%.

16. The composition according to claim 15, wherein the ratio of the concentration by weight of the at least one high-molecular-weight polyurethane to the concentration by weight of the at least one thickener, ranges from 0.05% to 20%.

17. The composition according to claim 16, wherein the ratio of the concentration by weight of the at least one high-molecular-weight polyurethane to the concentration by weight of the at least one thickener, ranges from 0.1% to 10%.

18. The composition according to claim 1, further comprising at least one adjuvant chosen from anionic, cationic, nonionic and amphoteric surfactants; perfumes; screening agents; preservatives; proteins; vitamins; provitamins; polymers other than the at least one high-molecular-weight polyurethane and different from the at least one thickener; vegetable, mineral and synthetic oils; polyols; silicones; and fatty alcohols.

19. The composition according to claim 18, wherein the polyols are chosen from glycols and glycerine.

20. A cosmetic method for the fixing and/or holding of a hairstyle comprising applying to hair a composition comprising, in a cosmetically acceptable medium,
at least one high-molecular-weight polyurethane with a number-average-molecular-weight ranging from 400,000 to 5,000,000 g/mol, and
at least one thickener,
wherein the cosmetic composition is provided in the form of a gel.
